(19) United States
(12) Patent Application Publication
(10) Pub. No.: US 2008/0200913 A1
(43) Pub. Date: Aug. 21, 2008

(54) Single Catheter Navigation for Diagnosis and Treatment of Arrhythmias

(76) Inventor: Raju R. Viswanathan, St. Louis, MO (US)

Correspondence Address: Bryan K. Wheelock, Suite 400, 7700 Bonhomme, St. Louis, MO, 63105, US

(21) Appl. No.: 12/023,020

(22) Filed: Jan. 30, 2008

Related U.S. Application Data
(60) Provisional application No. 60/900,078, filed on Feb. 7, 2007.

(51) Int. Cl. A61B 18/14

(52) U.S. Cl. 606/41

(57) ABSTRACT

A method of conducting a therapeutic ablation of cardiac tissue based upon local electrical signals in the tissue. The method includes navigating at least one electrophysiology catheter to a plurality of locations on the surface of the heart using a remote navigation system; sensing the electrical activity in the heart tissue at a plurality of the locations in the heart; and storing a value representative of the control variables of the remote navigation system at the location and a value representative of the electrical signal at the location; displaying a representation of at least some of the locations where the electrical activity was sensed on a display using graphic indicators of the timing of the sensed activity relative to the reference; determining the location at which the sensed electrical activity is earliest relative to a reference electrocardiogram signal; and automatically navigating an electrophysiology catheter to a position relative to selected location, and ablating tissue at the location with the electrophysiology catheter.

### CROSS-REFERENCE TO RELATED APPLICATION

This application claims priority to prior U.S. Patent Application Ser. No. 60/900,078, filed Feb. 7, 2007, the entire disclosure of which is incorporated herein by reference.

### FIELD

This invention relates to the diagnosis and treatment of cardiac arrhythmias, such as the diagnosis and treatment of supraventricular tachycardia (SVT).

### BACKGROUND

An arrhythmia is an abnormality or disturbance in the rate or rhythm of the heartbeat. Arrhythmias are caused by problems with the heart's electrical system, which alter the formation of the electrical impulse that begins a heartbeat or disrupt the pattern of conduction that distributes the impulse through the heart.

In the conventional treatment of arrhythmias, a plurality of catheters are individually navigated through the subject's vasculature, each of which is positioned in different locations in the subject's heart, to evaluate and determine the suspected site of the cause. Each of the plurality of catheters is used to measure the local electrical activity in the heart tissue in their respective locations. For example, in the case of diagnosing and treating a SVT, as many as five catheters are navigated into the heart, one to the coronary sinus, one to the HIS bundle, one to the high right atrium, one to the left atrium, and one to the right ventricle for pacing. The heart is paced from the catheter in the right ventricle, and electrical activity is measured at the other locations. The introduction of five catheters requires two punctures to be made in the left femoral artery, two punctures to be made in the right femoral artery, and one puncture to be made in an arm. There are risks associated with each puncture, as well as discomfort to the subject.

### SUMMARY

The present invention relates to methods for diagnosing and treating arrhythmias, such as SVT. In one embodiment, a method for diagnosing and treating arrhythmias, the method provides for utilizing at least one medical device to measure electrical activity at multiple locations on the cardiac tissue, rather than employing as many as five catheters that each measure electrical activity at a single location. The method further provides for conducting therapeutic ablation of cardiac tissue at desired locations based upon local electrical signals in the tissue. The method of navigating to different locations and measuring electrical activity at the different locations reduces the number of catheters that must be introduced into the body and navigated to the procedure site, and the potential for attendant complications.

### BRIEF DESCRIPTION OF THE DRAWINGS

FIG. 1 is a flow chart of a conventional five catheter procedure;

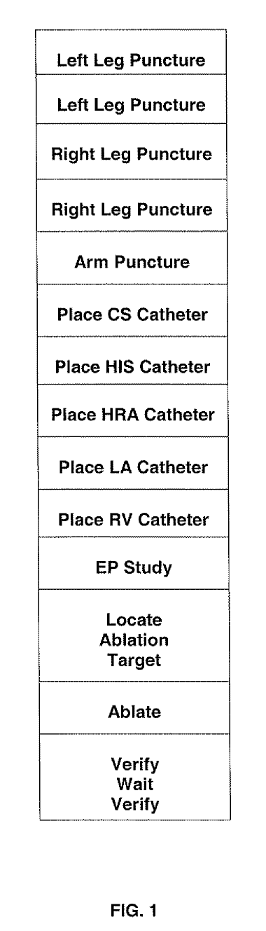

FIG. 2 is a flow chart of a preferred embodiment of a treatment procedure in accordance with the principles of this invention;

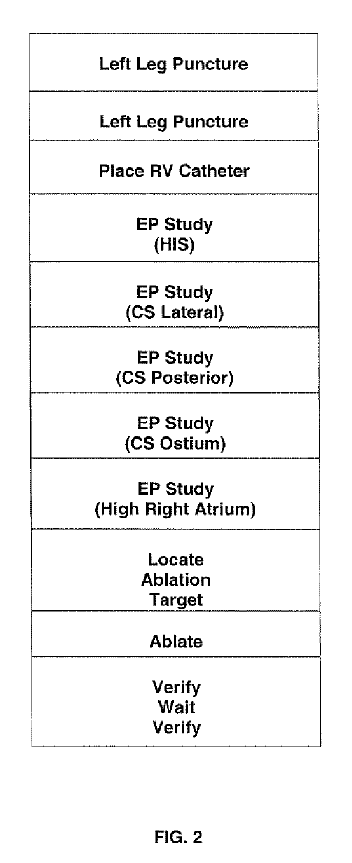

FIG. 3A is a diagram showing the navigation of an electrophysiology to the HIS bundle in accordance with the principles of this invention;

FIG. 3B is a diagram showing the navigation of an electrophysiology catheter to the coronary sinus lateral;

FIG. 3C is a diagram showing the navigation of an electrophysiology catheter to the coronary sinus posterior;

FIG. 3D is a diagram showing the navigation of an electrophysiology catheter to the coronary sinus ostium; and

FIG. 3E is a diagram showing the navigation of an electrophysiology catheter to the high right atrium.

Corresponding reference numerals indicate corresponding parts throughout the several views of the drawings.

### DETAILED DESCRIPTION OF THE EMBODIMENTS

The various embodiments of methods for diagnosing and treating arrhythmias in the present disclosure provide for conducting therapeutic ablation of cardiac tissue based upon local electrical signals in the tissue preferably using a remote navigation system.

In one embodiment, a method of conducting therapeutic ablation of cardiac tissue based upon local electrical signals in the tissue is provided that uses a remote navigation system. The method comprises navigating at least one medical device to different locations on the cardiac tissue in a successive manner, to sense electrical activity at each of the different locations on the cardiac tissue. The method determines the time differential of the sensed electrical activity relative to a reference point, for each of the different locations at which electrical activity is sensed. From the data, the method determines at least one location at which the sensed electrical activity is earliest relative to a reference point. The method then remotely navigates an electrophysiology medical device to the at least one determined location, and ablates the cardiac tissue at the at least one determined location.

Generally, the methods for diagnosing and treating arrhythmias comprise sensing the electrical activity in the cardiac tissue at a plurality of locations in the heart, determining the location or locations at which the sensed electrical activity occurs earliest relative to a reference point or time in the cardiac rhythm, remotely navigating an electrophysiology medical device to the determined location, and ablating tissue at the determined location. The step of sensing the electrical activity at various points comprises navigating at least one electrophysiology catheter to a plurality of locations and sensing the electrical activity at the plurality of locations with respect to a reference point within the cardiac rhythm. The remote navigation system can be a magnetic navigation system or other system for remotely orienting the distal end of a catheter.

These procedures can be facilitated by using unique ways of displaying sensed electrical activity obtained at multiple points, which can reduce the need for simultaneously measuring the electrical activity. In a preferred embodiment the sensed electrical activity is represented on a display. More preferably at least some of the points where the electrical activity was sensed are displayed using graphic indicators of the time of the sensed activity relative to a common reference point in time, such as a specific point within the cardiac rhythm. This facilitates the comparison of the signals gathered from different locations in a successive manner, and obviates the need for simultaneous measurements.

The reference point may be provided by an electrocardiogram signal, but could also be provided by an applied pacing signal. The measured electrical signals are preferably displayed in a manner that facilitates there interpretation. For example, at least some of the points where the electrical activity was sensed can be displayed on a display, such as a computer display, using graphic indicators of the time differential of the sensed activity relative to the reference point. Thus, the sensing of an electrical signal relative to a reference point at one location may be compared to subsequently sensed electrical signals at other locations relative to a reference point, such that the sensed signals at different locations may be compared without having to simultaneously measure electrical activity at the various location.

The remote navigation system may comprise a mechanical, electrostrictive, or other navigation system for remotely controlling the shape and orientation of the distal end of the device. These procedures can also be facilitated by the use of automated navigations systems, such as automated magnetic navigation systems, which allow a catheter to be automatically returned to a previous location, eliminating the need to leave a catheter at the measuring site in order to ablate tissues there. The remote navigation system can be a magnetic navigation system, in which case the control variables of the magnetic navigation system can include magnetic field direction and device length. Such systems are available from Stereotaxis, Inc., St. Louis, Mo.

The step of navigating an electrophysiology catheter to the determined location can comprise operating a remote navigation system to move the catheter, determining the location of the distal end of the catheter after it has been moved, and using the determined location as feed back in the control of the operation of the remote navigation system to navigate to the determined location, as well as to return to the determined location. Alternatively, the step of navigating an electrophysiology catheter to the determined location can comprise operating a remote navigation system by applying a control variable corresponding to the determined location to cause the electrophysiology catheter to move to the determined location.

Thus in preferred embodiment, a remote navigation system is used to navigate at least one electrophysiology catheter to a plurality of locations on the surface of the heart. At least one sensing catheter is used to sense electrical activity in the heart tissue at a plurality of the locations in the heart. A representation of at least some of the locations where the electrical activity was sensed may be displayed on a display using graphic indicators of the time of the sensed activity relative to the reference time. The approximate location or locations at which the sensed electrical activity is earliest relative to a reference electrocardiogram signal can accordingly be determined. An ablation catheter (which may be the same as the sensing catheter) is automatically navigated to a selected location relative to the determined location, and tissue at the location is ablated with the electrophysiology catheter. The selected location is the location where the sensed electrical signal had a predetermined relationship to the reference electrocardiogram signal.

In one embodiment of a method for diagnosing and treating arrhythmias, the remote navigation system stores at least one value representative of the location and at least one value representative of the electrical activity at the location, for each location that is sensed. The value representative of the location may be, for example coordinates in a reference frame, and the remote navigation system uses localization and the stored coordinates to navigate the electrophysiology catheter to the selected location. The value representative of the location may be, for example at least one control variable value of the remote navigation system at the location, and the remote navigation system uses the control variables to navigate the electrophysiology catheter to the selected location.

The display is preferably a two-dimensional display of the three dimensional operating region. Points where the electrical activity has been sensed are indicated in their relative locations. The relative timing of the electrical activity is preferably displayed as well. In this preferred embodiment the relative timing of the electrical activity relative to a common reference, such as an ECG signal, is displayed. This activity can be displayed using ordinal characters, such as numbers or letters, or it can be displayed using color coding, or symbols. In the case of ordinal characters, the graphic display includes ordinal characters indicating the locations in order from earliest to latest. The indicators can be color coded, with a color indicating a sequence of particular times or ranges of times.

Under the control of an automated navigation system the electrophysiology catheter can be automatically navigated to a position relative to selected location so that the tissue at the position can be ablated. This position can be the actual selected location, or it can be at a predetermined relative to the selected location and at least one other location, for example at a predetermined position relative to the location with the earliest signal and the location with the next earliest signal. After the catheter has been automatically navigated to the position, and before the ablation, the catheter can again sense the local activity to confirm the proper positioning of the catheter.

Accordingly, one embodiment of a method for navigating an electrophysiology catheter for conducting a therapeutic ablation of cardiac tissue based upon local electrical signals in the tissue is provided. The method comprises navigating at least one electrophysiology catheter in a successive manner to a plurality of locations on the surface of the heart using a remote navigation system, and sensing the electrical activity in the heart tissue at the plurality of locations on the surface of the heart in a successive manner. The method includes storing a value representative of the control variables of the remote navigation system for each location, and storing a value representative of the electrical signal relative to a reference electrocardiogram signal for each location. The method then displays a representation of at least some of the locations where the electrical activity was sensed on a display, using graphic indicators of the timing of the sensed activity relative to the reference. From the sensed data and indicators, the method allows for determining at least one location at which the sensed electrical activity is earliest relative to a reference electrocardiogram signal. The electrophysiology catheter is then automatically navigated to at least one position relative to at least one determined location, to ablate tissue at the at least one determined location. The steps of successive navigation and measurement or sensing of electrical activity are synchronized, such that upon navigating the electrophysiology catheter to a desired location the reference time is made available for use in determining the time of a sensed electrical signal relative to the reference. This timing data measured on the ECG system is made available to the navigation system, such that the navigation system may guide and advance the electrophysiology catheter to the next desired location in an efficient manner.

### Operation

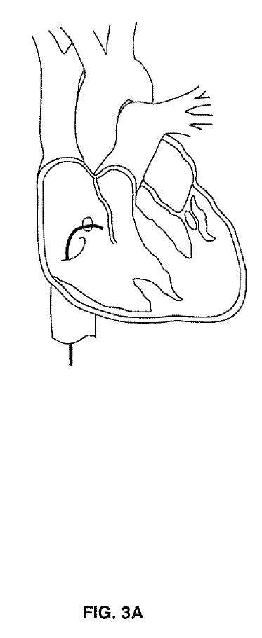

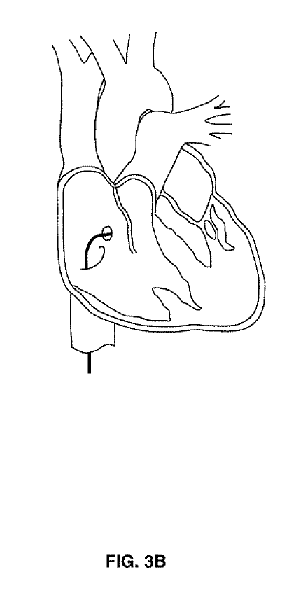

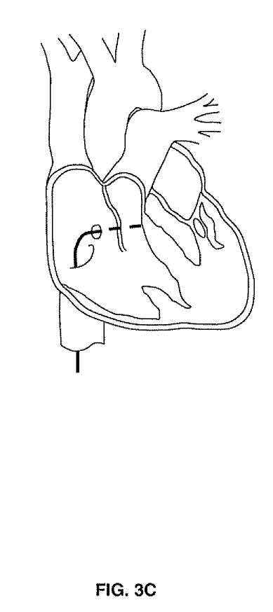

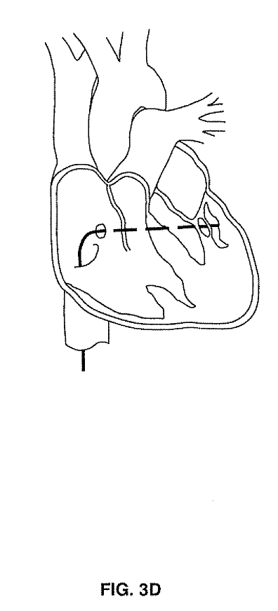

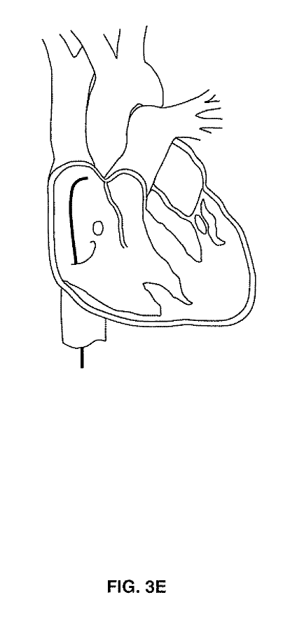

In a preferred embodiment of the methods of this invention, a pacing catheter is navigated to an appropriate location in the heart to apply a pacing signal to the heart, for example the right ventricle. The electrophysiology medical device or catheter is then navigated to a first location on the heart surface, such as the HIS bundle as shown in FIG. 3A, using fluoroscopy imaging to verify the position of the medical device. The Mitral preset position (at the 3 o'clock position) is selected and the Catheter Advancing System advances the electrophysiology catheter to different points to look for a HIS signal on an ElectroCardioGraph (ECG). Adjustments are made via the magnetic navigation system to obtain a good HIS electrical activity signal. The final location at which an HIS electrical signal is sensed is then stored. In addition, the navigation system may store an applying a control variable corresponding to the determined location, such as an applied magnetic field direction and strength for navigating the catheter to the location. A constellation of points are created to mark the HIS location, and the HIS constellation points are displayed on the fluoroscopic image. A pacing study is then performed at the HIS location, and the timing of sensed electrical signals is displayed on the fluoroscopic image. The measured electrical signal, relative to a reference such as a pacing signal or a reference electrocardiogram signal, is also stored for the HIS location.

The electrophysiology medical device or catheter is then retracted slightly, and navigated to another location on the heart surface, such as the Coronary Sinus Ostium as shown in FIG. 3B, using fluoroscopy imaging to verify the position of the medical device. Adjustments are made via the magnetic navigation system to obtain a good location for acquiring Coronary Sinus Ostium electrical signals. The final location at which the Coronary Sinus Ostium electrical signal is sensed is then stored.

The navigational vector is then changed to be more left lateral and the Catheter Advancing System advances the electrophysiology catheter into the Coronary Sinus Posterior, as shown in FIG. 3C. Fluoroscopy imaging may be used to verify the position of the electrophysiology catheter. Adjustments are made via the magnetic navigation system to obtain a good location for acquiring Coronary Sinus Posterior electrical signals. The final location at which the Coronary Sinus Posterior is contacted is then stored.

The navigational vector is then changed as needed to advance the electrophysiology catheter using the Catheter Advancing System into the Coronary Sinus Lateral, as shown in FIG. 3D. Adjustments are made via the magnetic navigation system to obtain a Coronary Sinus Lateral electrical activity signal. Fluoroscopy imaging may be used to verify the position of the electrophysiology catheter, and Left Anterior Oblique and Right Anterior Oblique X-rays are taken with the catheter in the Coronary Sinus Lateral. A desired constellation of points are marked along the Coronary Sinus Lateral on the X-ray image, and the resulting constellation is displayed on the fluoroscopy image. A pacing study is then performed at the Coronary Sinus Lateral location, and the timing of sensed electrical signals is displayed on the fluoroscopic image. The measured electrical signal, relative to a reference such as a pacing signal or a reference electrocardiogram signal, is stored for the Coronary Sinus Lateral location. The electrophysiology catheter is then retracted to the Coronary Sinus Posterior where a pacing study is performed, and the timing of sensed electrical signals at the Coronary Sinus Posterior is displayed on the fluoroscopy image. The electrophysiology catheter is then retracted to the Coronary Sinus Ostium where a pacing study is performed, and the timing of sensed electrical signals at the Coronary Sinus Ostium is displayed on the fluoroscopic image next to the points at which the timing data was taken.

The navigational vector is changed to be largely superior and the electrophysiology catheter is then navigated top the High Right Atrium, as shown in FIG. 3D. A pacing study is then performed at the High Right Atrium location, and the timing of sensed electrical signal, relative to a reference such as a pacing signal or a reference electrocardiogram signal, is stored and displayed on the fluoroscopic image. The measured timing data is recorded for each location, and is displayed on the fluoroscopic image next to the points at which the timing data was taken.

Based on the recorded timing data, and the ECG waves, a diagnosis is made and the target location representing the earliest electrical activity relative to a reference is determined. Magnetic direction is then changed to navigate the electrophysiology catheter to the determined target location. The ECG and pacing of the target location is performed for verification. Small adjustments may be made via the magnetic navigation system to explore the determined area for the site or location of the earliest activation. Once the site or determined location is selected, ablation of the selected location is performed. Pacing studies are subsequently performed to confirm that the ablation was successful in eliminating the early electrical activation at the site.

In some embodiments of the methods of this invention it may not be necessary to apply a pacing signal to the heart, so a pacing catheter may not be used. The electrophysiology catheter is used at each of the at least two sites to measure the electrical activity at each of the sites. Accordingly, at least one electrophysiology catheter is navigated to at least two sites in the operating region of the heart. The electrophysiology catheter is used at each of the at least two sites to measure the electrical activity at each of the sites. The electrophysiology catheter then performs pacing studies and ablation of at least one determined earliest electrical activation site. The at least one electrophysiology catheter may be similar in construction, or identical to, the pacing catheter, and in fact the same catheters may be used for both pacing and for measuring local electrical activity.

The successively measure electrical activity may be displayed to the user in manner that allows the user to determine their relative priority. The points can be displayed on a map or display representation of the operating region with ordinal characters (e.g. numbers, letters, symbols, or graphics which indicate the order or sequence of the electrical activity. The points can also be displayed using a color coding system such as varying intensity or different colors. The points can also be in a flashing or changing sequence to indicate the order or sequence of the electrical activity. Of course any other manner of indicating to the viewer the relative order or sequence of the signals can be used.

The measured electrical signals are preferably also displayed in graphic form for example as an ECG traces. These ECG traces are preferably aligned in the same time scale to readily indicate their relative order or sequence. These ECG traces can be identified with their corresponding points on the display. For example each ECG trace can be labeled with a numeral, letter, symbol or graphic associated with its corresponding point. Alternatively, each ECG trace may be framed in a color associated with its corresponding point.

The ECO traces are preferably presented in a column, arranged in order with the earliest signal on top. This arrangement helps the user identify the earliest signal points to identify the appropriate locations for therapeutic ablation. An ablation catheter, which can be the pacing catheter, or one of the at least one electrophysiology catheters can then be navigated to the appropriate site (typically the site of the earliest signal) to therapeutically ablate the local tissue to interfere with the disruptive electrical activity.

The advantages of the above described embodiment and improvements should be readily apparent to one skilled in the art, as to enabling the navigation of medical devices within a subject using remote navigation systems. Additional design considerations may be incorporated without departing from the spirit and scope of the invention. Accordingly, it is not intended that the invention be limited by the particular embodiment or form described above, but by the appended claims.

What is claimed is:

1. A method of conducting a therapeutic ablation of cardiac tissue based upon local electrical signals in the tissue using a remote navigation system, the method comprising:
navigating at least one medical device to different locations of cardiac tissue in a successive manner to sense electrical activity at the different locations on the cardiac tissue;
determining the time differential of the sensed electrical activity relative to a reference point for each of the different locations;
determining at least one location at which the sensed electrical activity is earliest relative to a reference point;
remotely navigating an electrophysiology medical device to the at least one determined location; and
ablating the cardiac tissue at the at least one determined location.

2. The method according to claim 1 wherein the step of sensing the electrical activity comprises navigating at least one electrophysiology catheter to a plurality of different locations in a successive manner, and sensing the electrical activity at each of the plurality of locations relative to a reference point.

3. The method according to claim 1 wherein the step of navigating an electrophysiology catheter to the determined location comprises operating the remote navigation system to move the catheter, determining the location of the distal end of the catheter, and using the determined location as feed back in the control of the operation of the remote navigation system to return to the determined location.

4. The method according to claim 1 wherein the step of navigating an electrophysiology catheter to the determined location comprises operating a remote navigation system by applying a control variable corresponding to the determined location.

5. The method according to claim 4 wherein the remote navigation system is a magnetic navigation system, and wherein the control variables of the magnetic navigation system include magnetic field direction and device length.

6. The method according to claim 1 wherein the reference is an electrocardiogram signal.

7. The method according to claim 1 further comprising representing on a display at least some of the points where the electrical activity was sensed, using graphic indicators of the time of the sensed activity relative to the reference.

8. A method of conducting a therapeutic ablation of cardiac tissue based upon local electrical signals in the tissue using a remote navigation system, the method comprising:
using a remote navigation system to navigate at least one electrophysiology catheter to a plurality of locations on the surface of the heart;
sensing the electrical activity in the heart tissue at a plurality of the locations in the heart;
determining the location at which the sensed electrical activity is earliest relative to a reference electrocardiogram signal;
displaying a representation of at least some of the locations where the electrical activity was sensed on a display using graphic indicators of the time of the sensed activity relative to the reference;
automatically navigating an electrophysiology catheter to a selected location, and ablating tissue at the location with the electrophysiology catheter.

9. The method according to claim 8 wherein the selected location is the location where the sensed electrical signal had a predetermined relationship to the reference electrocardiogram signal.

10. The method according to claim 7 wherein the remote navigation system stores at least one value representative of the location and at least one value representative of the electrical activity at the location, for each location.

11. The method according to claim 10 wherein the value representative of the location are coordinates in a reference frame, and wherein the remote navigation system uses localization and the stored coordinates to navigate the electrophysiology catheter to the selected location.

12. The method according to claim 10 wherein the value representative of the location is at least one control variable value of the remote navigation system at the location, and wherein the remote navigation system uses the control variables to navigate the electrophysiology catheter to the selected location.

13. The method according to claim 8 wherein the graphic display includes ordinal characters indicating the locations in order from earliest to latest.

14. The method according to claim 8 wherein the graphic display includes color coding indicating locations within predetermined ranges of time.

15. A method of conducting a therapeutic ablation of cardiac tissue based upon local electrical signals in the tissue, the method comprising:
navigating at least one electrophysiology catheter to a plurality of locations on the surface of the heart using a remote navigation system;
sensing the electrical activity in the heart tissue at one or more of the plurality of the locations in the heart;
displaying a representation of at least some of the locations where the electrical activity was sensed on a display using graphic indicators of the time of the sensed activity relative to the reference;
determining at least one location at which the sensed electrical activity is earliest relative to a reference electrocardiogram signal;
automatically navigating an electrophysiology catheter to a position relative to determined location, and ablating tissue at the location with the electrophysiology catheter.

16. The method according to claim 15 wherein the position is at a predetermined position relative to the location with the earliest signal and the location with the next earliest signal.

17. The method according to claim 15 further comprising sensing the electrical activity in the heart tissue at the position to confirm that the catheter has been navigated to the position.

18. A method of conducting a therapeutic ablation of cardiac tissue based upon local electrical signals in the tissue, the method comprising:
navigating at least one electrophysiology catheter in a successive manner to a plurality of locations on the surface of the heart using a remote navigation system;
sensing the electrical activity in the heart tissue at one or more of the plurality of locations in the heart in a successive manner; and storing a value representative of the control variables of the remote navigation system at each location and a value representative of the electrical signal relative to a reference electrocardiogram signal at each location;
displaying a representation of at least some of the locations where the electrical activity was sensed on a display using graphic indicators of the timing of the sensed activity relative to the reference;
determining at least one location at which the sensed electrical activity is earliest relative to a reference electrocardiogram signal;
automatically navigating an electrophysiology catheter to a position relative to the at least one determined location, and ablating tissue at the at least one determined location with the electrophysiology catheter.

19. The method according to claim 18 wherein the position is the location with the earliest time relative to the reference electrocardiogram signal, and wherein the remote navigation system uses the stored value representative of the control variables for that location to navigate the electrophysiology catheter to that position.

20. A method of conducting a therapeutic ablation of cardiac tissue based upon local electrical signals in the tissue, the method comprising:
navigating at least one electrophysiology catheter to a plurality of locations on the surface of the heart using a remote navigation system;
sensing the electrical activity in the heart tissue at one or more of the plurality of locations in the heart;
displaying a representation of at least some of the locations where the electrical activity was sensed on a display using graphic indicators of the time of the sensed activity relative to the reference;
determining the location at which the sensed electrical activity is earliest relative to a reference electrocardiogram signal;
automatically navigating an electrophysiology catheter to a position relative to a selected location, and ablating tissue at the location with the electrophysiology catheter.
